(19) United States
(12) Patent Application Publication
(10) Pub. No.: US 2010/0310622 A1
(43) Pub. Date: Dec. 9, 2010

(54) DRY EYE TREATMENT BY PUNCTA PLUGS

(75) Inventors: Anuj Chauhan, Gainesville, FL (US); Heng Zhu, Tonawanda, NY (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF FLORIDA RESEARCH FOUNDATION, INC., GAINESVILLE, FL (US)

(21) Appl. No.: 12/745,829

(22) PCT Filed: Dec. 17, 2008
(86) PCT No.: PCT/US08/87176

§ 371 Date: Aug. 20, 2010

Related U.S. Application Data
(60) Provisional application No. 61/007,859, filed on Dec. 17, 2007.

(51) Int. Cl. A61F 2/00; A61K 38/13; A61K 31/5377; A61P 27/02

(52) U.S. Cl. 424/422; 514/20.8; 514/236.2

(57) ABSTRACT

A punctal plug and method of treating dry eyes are provided. The punctal plug has two or three layer structure and contains at least one drug, for treating conditions such as dry eyes contained in a core, a potion of which is covered by a drug impermeable shell such that drug can radially diffuse from the core. The punctal plug can be inserted into a patient's upper punctum, lower punctum, or both to deliver the drug for an extended period of time. The drug for treating dry eyes can be, for example, cyclosporine A. The plug can also be used for extended delivery of ophthalmic drugs.

### CROSS-REFERENCE TO RELATED APPLICATION(S)

The present application claims the benefit of 35 U.S.C §111(b) of U.S. Provisional Patent Application Ser. No. 61/007,859, filed Dec. 17, 2007, which is hereby incorporated by reference herein in its entirety, including any figures, tables, or drawings.

### BACKGROUND OF THE INVENTION

It is estimated that 10% to 30% of people suffer from the condition known as dry eyes. Dry eye refers to an ocular affliction characterized by a dryness sensation in the eye accompanied by grittiness, tearing, burning, blurred vision, and a foreign-body sensation. If left untreated, dry eyes can lead to more serious problems, such as dry eye syndrome or, in extreme cases, blindness. It is generally accepted that dry eyes are caused by an abnormality in the quality or quantity of tears on the eye surface, such as tear imbalance that could lead to a loss of proper lubrication, leading to discomfort. Tears protect our eyes from any kind of external stimuli and the drying of eyes can ultimately lead to inflammation of ocular surface and epithelial cell damage, which in turn reduces the production of tears or mucus leading to a further decrease in both the quality and quantity of tears.

The tear film consists of three layers: the lipid layer, the aqueous layer, and the mucus layer. The lipid layer is in contact with the air, and functions to inhibit evaporation of tears. The middle layer is the aqueous layer, containing ions and larger molecules such as proteins. The mucus layer is between the aqueous layer and the ocular epithelial cells and helps stabilize the tear film. Aqueous tears are produced by the lacrimal glands and the conjunctiva, and those that remain in the eye can be evaporated or drained through the lacrimal canaliculi into the nose. Understanding the mechanisms and dynamics of tear production and elimination is important for developing dry eye treatments. A reduction in tear production or increase in tear elimination will often lead to dry eye. In addition, the lipid layer and the mucus layer play important roles in the dynamics of aqueous tears. For example, imperfections of the lipid layer can increase tear evaporation rates, and insufficient production of mucus can destabilize the tear film and lead to tear film rupture. Therefore, to treat dry eye, it is advantageous to improve the quality and quantity of aqueous tears, as well as the mucus and lipid layers.

Ocular conditions, such as dry eyes, are generally treated by topical application of drugs. Eye drops have been the traditional method of delivering ophthalmic drugs. However, the retention time of drugs delivered via eye drops with the ocular surface is typically only a few minutes due to how quickly tears are refreshed. The drugs are then drained into the nose through lacrimal canaliculi, or lost through other means such as evaporation or transport across the ocular epithelia. Additionally, the ocular surface usually has low permeability to the drugs. Due to these factors, using eye drops often results in low bioavailability, and it is estimated that typically less than 5% of the instilled drug enters the eye. A large portion of the instilled drug is drained into the nose and is taken up systemically, which may cause serious side effects. Therefore, it is important to increase the bioavailability of the drugs and reduce drug wastage by reducing drug elimination into the nose.

Several ways of increasing the retention time and the bioavailability of ophthalmic drugs have been proposed. One such method has been to increase the viscosity of the instilled fluid. It has been shown that by increasing the viscosity from about one centipoise (cp), which is the viscosity of water at room temperature, to about 60 cp can increase the retention time several times over, which leads to higher bioavailability. However, instilling fluid of high viscosity can lead to discomfort in the patient due to excessive shearing between the eyelids and the fluid during blinking.

Another proposed method has been to use controlled delivery devices such as contact lenses. For example, nanoparticle-laden contact lenses for controlled delivery of ophthalmic drugs have been proposed (U.S. Patent Application No. 2004/0241207). The contact lenses may be able to increase the bioavailability of the drugs to as much as 40% and the delivery period may be as long as several weeks. However, many patients, especially dry eye patients, have poor tolerance for contact lenses. For patients with dry eyes, their ocular surface epithelium may be damaged, and the extra shearing provided by contact lenses can cause further discomfort and can hinder the healing of the epithelium.

The use of punctal plugs has also been proposed as a method of increasing the bioavailability and reducing the wastage of ophthalmic drugs. Punctal plugs have essentially cylindrical shapes and can be made of metals or polymers such as poly hydroxyl ethylmethacrylate (p-HEMA), silicone, or hydrogel. Doctors can insert a punctal plug into a patient's punctum, which is an opening of the lacrimal canaliculi. According to anatomical studies, each canaliculus has a vertical part that is about 2 mm long and a horizontal part that is about 10 mm long. The diameter of the vertical and the horizontal parts are about 0.3 mm and 0.5 mm, respectively. The joint between these two parts is called the ampulla and its diameter can be up to 2 to 3 mm The commercial punctal plugs range in length from 1.1 to about 2 mm and in diameter from 0.4 to 1.1 mm The punctal plugs are inserted into the vertical portion of the canaliculi. Typical drug eluding punctal plug designs described in patent literature consist of cylindrical cores coated with an impermeable shell to minimize the drug loss into the cananliculus tissue. In such devices, the drug essentially diffuses out from the circular cross-section in contact with the tears. A punctal plug can be worn for long periods of time, generally over a month. Standard punctal plugs have been shown to help treat severe dry eyes, but they only do so by increasing the volume of tears on the eye surface. The punctal plugs block the flow of the tears from the eyes to the nose through the canaliculi. However, many other factors often contribute to dry eyes, including abnormity of tear osmolarity, inflammation of the ocular surface, and epithelial cell damage. These problems cannot be adequately treated by only increasing tear volume. Epithelial cell damage and ocular surface inflammation can reduce the production of tears or mucus and thus further decrease both the quality and quantity of tears. Therefore, punctal plugs are sometimes used in conjunction with other treatments, such as instilling artificial tears or eye drops that can reduce inflammation.

Punctal plugs that can also deliver dry eye medication can help treat dry eyes merely by inserting the plugs. Thus, there exists a need in the art for an efficient punctal plug that can deliver dry eye medication or other medications at a sustained rate for an extended period of time.

### BRIEF SUMMARY OF THE INVENTION

Embodiments of the invention provide punctal plugs, methods of their preparation and methods for treatments such as the treatment of dry eyes. The punctal plugs contain, and serve as delivery vehicles for, drugs and/or other agents, such as nutritional supplements and lubricants. The rate of drug release from the punctal plugs can be zero order or a higher order.

The punctal plug comprises a drug contained within a solid core, and a solid shell, for example, of a substantially cylindrical shape surrounding a portion of the core radially and, optionally, on one axial end. The shell can be effectively impermeable with respect to the drug. The shape can be any shape that permits a directed delivery of drugs to the tears or the eye surface and allows for the inserting and securing of the punctal plug in the lacrimal canaliculi. For a cylinder design, the core of the plug can be enclosed on the radial sides of the plug or can have the core exposed at a single site effectively directed toward the eye. The plugs can vary significantly in dimension. For a cylindrically shaped embodiment, the diameter of the plug can be as small as about 0.4 mm to as large as about 2 mm, and the length of the plug can be of about 1.1 mm to about 5 mm Plugs that are longer than 2 mm must be sufficiently soft and flexible so as to curve into the shape of the canaliciulus during insertion. The core can be attached to, or physically restricted within, the shell of the plug. The shell can be on only a portion of the radial sides such that the drug can diffuse radially into the tear fluid. The plug can also contain at least one highly permeable layer within the shell and around the drug supplying core to augment the rate of delivery of the drug from the punctal plug based on the absolute and relative affinities and permeabilities of the core and the highly permeable layer.

The core material is selected to have a desired affinity and permeability to the drugs or other agent incorporated therein. The solid nature of the core is derived from polymers, which may be cross-linked into networks. In general, the permeability should be relatively high to allow transport of the drug from the core. The affinity for the drug, in conjunction with the permeability, allows for a desired release rate of the drug from the punctal plug. Materials that provide the desired affinities and permeabilities will depend upon the nature of the drug. The permeabilities and affinities of a particular core can be modified by the presence of a relatively small molecule additive to the core polymer that is compatible with the eye. For example, vitamin E can be used as an additive with a hydrophobic polymer core. Silicone hydrogels can be used as the core material. Hydrophilic and/or hydrophobic drugs can be included in silicone hydrogel cores. Polymers used for the cores can be modified for specific interactions with particular drugs. Surfactants can be incorporated into the core to increase or decrease the release rates of the drugs depending on the relative interactions between various components of the plug, and the interactions with the tear fluid. Nano- or Micro-particles can be included within the core, where the particles contain the drug and release it from the core. The core material can be bioabsorbable and the rate of release of the drug can be the rate of bioabsorption of the core or greater. The core can be partitioned or shaped in any fashion to enhance the surface area for exchange of the drug between the core material and a highly permeable material (layer) in contact with the core or with the tear fluid in contact with the plug.

The shell is substantially impermeable to the drug, such that the drug is directed to release by the core in a portion that is not enclosed by the shell. The shell can be either a rigid material or an elastomeric material. The shell can be either non-bioabsorbable or bioabsorbable. When both the core and the shell are bioabsorbable, the removal of the plug may not be needed during a treatment protocol using the novel punctal plugs.

A method of preparing the punctal plugs of the invention involves providing the core and the shell and loading the core with the drug or other agents for treatment of the eye. The core can be formed by polymerization of at least one monomer. The shell can be formed by polymerization of at least one monomer. The core can be formed or placed within a preformed shell, or the shell can be formed or placed around a preformed core. The formation of a shell about a preformed core can be by coating, casting or molding around the solid core. The drug can be loaded by inclusion in the monomer mixture or it can be loaded from solution after polymerization of the core. The drug can be loaded into the core either before or after fixing the core in the shell. The solution can be aqueous or non-aqueous.

A method of treating dry eyes comprises the insertion of a punctal plug into a patient's upper punctum, lower punctum, or both and allowing the punctal plug to deliver a drug to the eye. The punctal plug can deliver a desired amount of the drug(s) per day, with release rates of up to, for example, about 50 μg/day, and can be left in for an extended period of time, such as about a month or more. Additionally, the drug can be any suitable drug for treatment of or through the eye. For example, cyclosporine A can be delivered by the punctal plugs for the treatment of dry eyes.

### BRIEF DESCRIPTION OF THE DRAWINGS

FIG. 1 is a view showing the upper punctum, lower punctum, and lacrimal canaliculi.

FIG. 2 is a view showing a punctal plug inserted into the lower punctum.

FIG. 3 is a cross sectional view and an end view of a punctal plug with a core and a partial drug impermeable shell according to an embodiment of the invention where the end of the cross section view intended to be placed distal to the eye is adjacent to the end view.

FIG. 4 is a cross sectional view and an end view of a punctal plug with a core, a high drug permeable second shell and a drug impermeable shell according to an embodiment of the invention where the end of the cross section view intended to be placed proximal to the eye is adjacent to the end view.

FIG. 5 is a cross sectional view and an end view of a punctal plug with a core, a high drug permeable second shell and a drug impermeable shell covering a portion of the second shell according to an embodiment of the invention where the end of the cross section view intended to be placed distal to the eye is adjacent to the end view.

FIG. 6 is a cross sectional view and an end view of a punctal plug with a core separated by a void on its sides from a shell according to an embodiment of the invention where the end of the cross section view intended to be placed proximal to the eye is adjacent to the end view.

FIG. 7 is a cross sectional view and an end view of a punctal plug with a core partitioned into four columns surrounded by a high drug permeable second shell and a drug impermeable shell according to an embodiment of the invention where the end of the cross section view intended to be placed proximal to the eye is adjacent to the end view.

FIG. 8 is a cross sectional view and an end view of a punctal plug with a core partitioned into spherical portions and a shell with a circular opening that is smaller than the diameter of the spheres according to an embodiment of the invention where the end of the cross section view intended to be placed proximal to the eye is adjacent to the end view.

FIG. 9 is a graph showing a drug release profiles from three punctal plugs of the structure in FIG. 3 with exposed lengths of 2.93 mm, 5.03 mm, and 7.17 mm respectively.

FIG. 10 is a graph showing a drug release profiles from punctal plugs of the structure in FIG. 3 with physiological compatible dimensions.

FIG. 11 is a graph showing a drug release profiles from punctal plugs of the structure in FIG. 3 with physiological compatible dimensions containing drug loadings of 30% and 40% respectively.

FIG. 12 is a graph showing a drug release profiles from punctal plugs of the structure in FIG. 5 with exposed lengths of 2.95 mm, 5.05 mm, and 7.27 mm respectively.

FIG. 13 is a graph showing a drug release profiles from punctal plugs of the structure in FIG. 4 for two different lengths.

FIG. 14 is a graph showing a drug release profiles from punctal plugs of the structure in FIG. 4 for different drug loadings.

### DETAILED DESCRIPTION OF THE INVENTION

Embodiments of the invention are directed to punctal plugs for the delivery of ophthalmic drugs including dry eye medicine and methods of treating dry eyes using punctal plugs. Advantageously, the punctal plugs can be used to deliver drugs at a nearly constant rate for long periods of time.

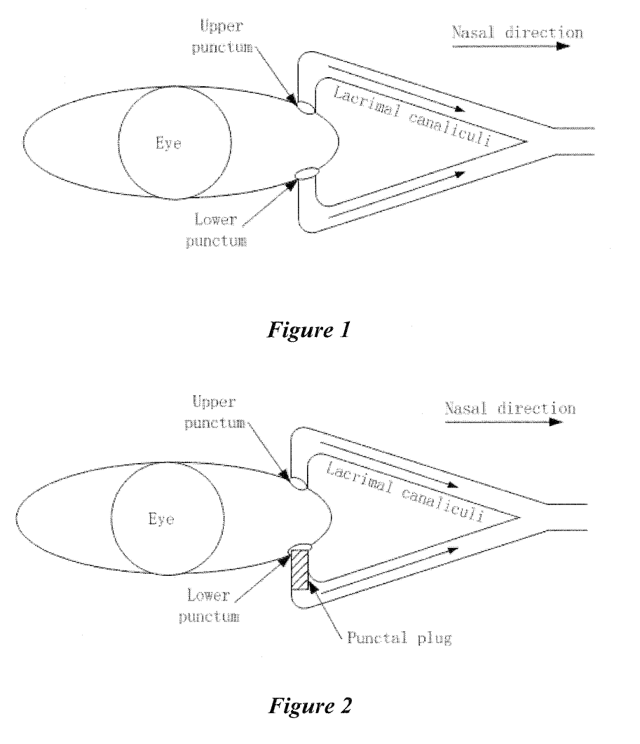

Referring to FIG. 1, an important route of tear elimination from the eye surface is drainage into the nose through the lacrimal canaliculi. Therefore, blocking the lacrimal canaliculi using punctal plugs can help increase tear volume. However, since dry eyes are usually caused by many factors, such as abnormality of tear osmolarity, inflammation of the ocular surface, and epithelial cell damage, it is preferable to use some form of dry eye medication when treating dry eyes. Thus, the present invention pertains to the use of punctal plugs that can deliver drugs to the eye, in addition to blocking tear drainage through the lacrimal canaliculi.

Referring to FIG. 2, a punctal plug can be inserted into the lacrimal canaliculi to block tear drainage. Compared to existing topical treatments, such as eye drops, punctal plugs of the present invention can increase the bioavailability of dry eye treatment drugs since the punctal plugs block tear drainage, which is considered a major route of drug loss from the eye surface.

Punctal plugs can typically be worn for extended periods of time, such as a month or more. A visit to a clinic may be needed to insert and remove the plugs. Thus, in an embodiment of the invention, a punctal plug is able to deliver an ophthalmic drug for an extended period of time, for example, about a month or more. Advantageously, employing this embodiment, dry eye patients may not need to apply eye drops or otherwise administer drugs on their own, which can avoid the possible problem of poor patient compliance.

In an embodiment of the invention, the punctal plug has a substantially cylindrical shape. Thus, the punctal plug is generally cylindrical but may have minor changes in its shape that preclude it from being a perfect cylinder. Other embodiments of the invention can employ shapes that allow appropriate placement, orientation and sealing of the lacrimal canaliculi.

A punctal plug of the present invention is constructed to allow placement into the upper or lower punctum of a patient. Punctal plug for humans are specifically exemplified herein. For example, the punctal plug can be substantially cylindrical with a diameter of about 1 millimeter (mm) and a length of about 2 mm. However, the actual size or shape of a punctal plug can vary according to the situation and be suitable for a particular patient.

In an embodiment of the invention, the drug delivered by the punctal plug is cyclosporine A. Cyclosporine A is a cyclic polypeptide consisting of 11 amino acids that is often used to treat dry eyes. When applied on the ocular surface, cyclosporine A is believed to inhibit T cell activation and therefore increase tear production. It is also believed to increase the number of mucin-secreting goblet cells. Due to its low solubility in water, delivery of cyclosporine A by simple aqueous solution can lead to low bioavailability and potential side effects from drainage of the drug solution into the nose. Other delivery methods, such as ointments, have also been used, but can cause discomfort due to excessive shearing. Currently, cyclosporine A is commercially available in the form of an emulsion in aqueous eye drops, such as RESTASIS®. While it has been proven to relieve dry eye symptoms, the exact mechanism is not clear. According to the instructions of RESTASIS®, the daily dose of cyclosporine A by eye drops should be about 20-30 μg. Assuming a common bioavailability of about 1%, the therapeutic requirement of the drug is about 0.2-0.3 μg/day. The bioavailability of cyclosporine A delivered by a punctum plug can be larger than that delivered by eye drops, which suffers due to tear drainage. Therefore, in an embodiment, a punctal plug of the present invention can deliver cyclosporine A to the eye at a minimum rate of 0.2 μg/day, to a rate of about 10 μg/day and to a rate of as much as 50 μg/day, depending on the bioavailability of the specific design used.

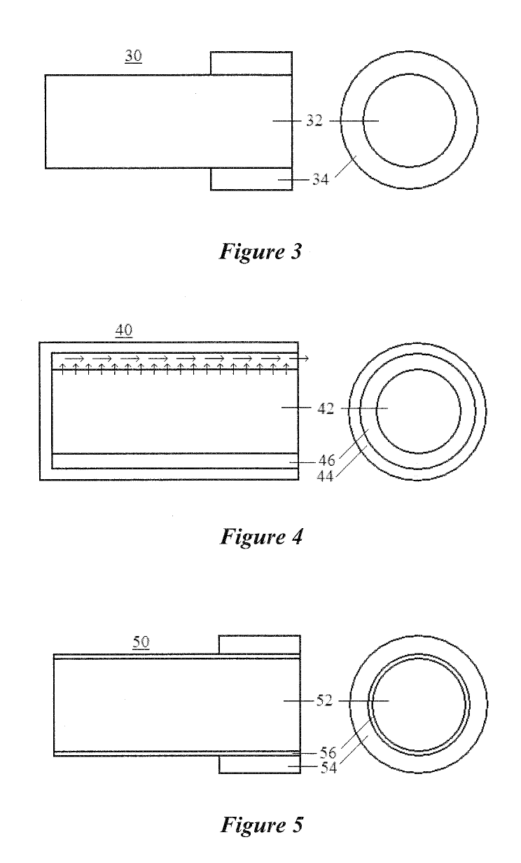

Referring to FIG. 3, in an embodiment of the invention, a punctal plug 30 can have a core-shell design with a core 32 containing a drug, such as cyclosporine A, that is to be delivered to a patient, and a shell 34 around a portion of the core. The core 32 can be made of a material that is permeable to the drug, and the shell 34 can be made of a material that is impermeable to the drug. Since after a punctal plug is inserted, a portion of the surface of the plug is in contact with the inner wall of the lacrimal canaliculi, which contain blood vessels, potentially any drug that is released in the radial direction can be absorbed by the canaliculus wall and enter the blood. Therefore, the release of drug should be biased toward the end of the punctal plug that is in contact with tears near the eye. The drug release from a circular end of the plug is often negligible when not in contact with any tissue and/or tear fluid. Therefore, in some embodiments, as shown, it is not necessary to coat that end opposite the eye when inserted with an impermeable layer. In one embodiment, the shell can enclose the plug at a single end to direct drug release toward the eye at the end free of the shell. As shown in FIG. 3, a cylindrical plug can have a shell on a portion of the outside of the cylinder with no shell on either ends of the cylindrical plug. The shell is only on a portion of the core, but is sufficient to promote extension of the core from the tissue of the punctal walls and allow radial diffusion of the drug from the core into the tear fluid. The puntal plugs can have any shape that allows the plug to be positioned and secured in the punctum.

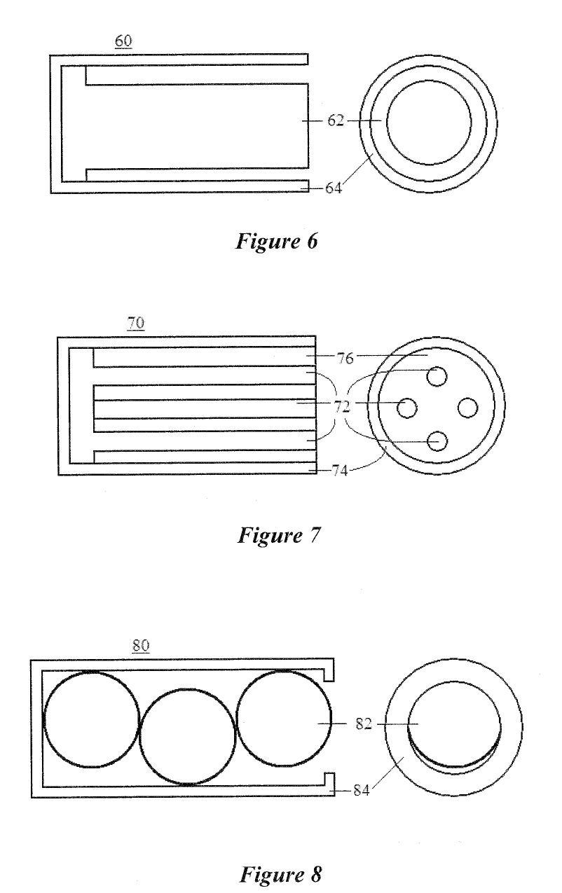

The core containing the drug can be cross-linked where a permeable core material, such as hydroxyl ethylmethacrylate (HEMA), is cross-linked with a cross-linker such as ethylene glycol dimethacrylate (EGDMA). Both the permeable and impermeable materials are biocompatible. For a punctal plug as shown in FIG. 3, since only a fraction of the core is covered with the shell, the drug can diffuse out from the end of the core and from the exposed curved radial surface. The residence time of the drug released by the plug from the radial surface depends on the diameter of the core. The release from the radial surface can be varied to optimize a desired release rate. Once the drug is released from the core radially into the surrounding tears inside the canaliculus (or a high permeability annulus as is disclosed below in other embodiments of the invention), the drug will diffuse axially into the tears. The axial diffusion time is short because of the high drug diffusivity in tears (or the high permeability annulus) compared to the diffusivity in the core. To avoid the drug released radially into the tears from entering the canaliculus tissue, an impermeable shell can be used as shown in FIG. 6. When the release occurs only from the end in contact with the tears, the release rates depends strongly on the degree of mixing with the tear fluid in the canthus region, and can be detrimentally impacted by protein binding to the cross-section. Protein binding will have less of an impact on plugs in which the drug diffuses mostly radially out of the core and then axially through the tear filled canaliculi. The release rate from the plug can be controlled by changing the cross-linking of the core.

The shell and the core can be made of any suitable punctal plug materials known in the art, such as metals, polymers, or silicon hydrogels. In an embodiment, the shell can be made of poly ethylene glycol dimethacrylate (p-EGDMA) while the core can be made of poly hydroxyl ethylmethacrylate (p-HEMA) and can contain cyclosporine A or other drugs to be delivered. The polymer p-HEMA is permeable to cyclosporine A. The core is solid in the sense that it reside within and can extend from the shell without flowing under the force of gravity unless the solid dissolves or degrades. For some embodiments of the invention, the solid core may be an extremely viscous liquid at body temperature. The shell is a solid material in the traditional sense of the term solid and will maintain the general shape of the punctal plug during storage and insertion of the plug. The release rate of the drug is controlled by axial diffusion through the shell-free end of the core that is in contact with tissue and/or tears. This can potentially lead to a non-zero-order release rate, which can be desirable. For example, when the therapy requires a slow decrease in the delivery of the drug over time, a higher order release rate can be preferred. In an alternate embodiment, the shell could be made of a material that has a very low permeability for the drug. The permeability will depend on the drug and the material chosen for the plug. For example, a polydimethylsiloxane (PDMS) network can be used as a low permeable shell for a plug to deliver cyclosporine A.

In one embodiment, the core is a silicone hydrogel. Suitable silicone hydrogel materials include, without limitation, silicone hydrogels made from silicone macromers such as the polydimethylsiloxane methacrylated with pendant hydrophilic groups described in U.S. Pat. Nos. 4,259,467; 4,260,725 and 4,261,875; or the polydimethylsiloxane macromers with polymerizable functional described in U.S. Pat. Nos. 4,136,250; 4,153,641; 4,189,546; 4,182,822; 4,343,927; 4,254,248; 4,355,147; 4,276,402; 4,327,203; 4,341,889; 4,486,577; 4,605,712; 4,543,398; 4,661,575; 4,703,097; 4,740,533; 4,837,289; 4,954,586; 4,954,587; 5,034,461; 5,070,215; 5,260,000; 5,310,779; 5,346,946; 5,352,714; 5,358,995; 5,387,632; 5,451,617; 5,486,579; 5,962,548; 5,981,615; 5,981,675; and 6,039,913. The silicone hydrogels can also be made using polysiloxane macromers incorporating hydrophilic monomers such as those described in U.S. Pat. Nos. 5,010,141; 5,057,578; 5,314,960; 5,371,147 and 5,336,797; or macromers comprising polydimethylsiloxane blocks and polyether blocks such as those described in U.S. Pat. Nos. 4,871,785 and 5,034,461.

The silicone containing monomers that may be in the formulation of a silicone hydrogel core of the present invention can be oligosiloxanylsilylalkyl acrylates and methacrylates containing from 2-10 Si-atoms. Typical methacrylate representatives include: tris(trimethylsiloxysilyl)propylmethacrylate, triphenyldimethyldisiloxanylpropylmethyl-methacrylate, pentamethyldisiloxanylpropylmethylmethacrylate, tert-butyl-tetramethyldisiloxanyl-ethylmeth-acrylate, methyldi(trimethylsiloxy)silylpropyl-glyceryl methacrylate; pentamethyldisiloxanylpropylmethylmethacrylate; heptamethylcyclotetrasiloxanyl-propylmethyl methacrylate; and undecamethylpentasiloxanylpropylmethacrylate.

Other representative silicon-containing monomers which may be used for the preparation of silicone hydrogel cores of the present invention includes silicone-containing vinyl carbonate or vinyl carbamate monomers such as: 1,3-bis[4-vinyloxycarbonyloxy) but-1-yl]tetramethyldisiloxane; 3-(trimethylsilyl)propyl vinyl carbonate; 3-(vinyloxy-carbonylthio)propyl[tris(trimethylsiloxy)]silane; 3-[tris(trimethylsiloxy)silyl]propyl vinyl carbamate; 3-[tris(trimethylsiloxy)silyl]propyl, allyl carbamate; 3-[tris(trimethylsiloxy)-silyl]propyl vinyl carbonate; t-butyldimethylsiloxethyl vinyl carbonate; trimethylsilyiethyl vinyl carbonate; and trimethylsilylmethyl vinyl carbonate. Polyurethane-polysiloxane macromonomers (also sometimes referred to as prepolymers), which have hard-soft-hard blocks like traditional urethane elastomers may be used. Examples of such silicone urethanes which may be included in the formulations of the present invention are disclosed in a variety or publications, including Lai, Yu-Chin, “The Role of Bulky Polysiloxanylalkyl Methacrylates in Polyurethane Polysiloxane Hydrogels,” Journal of Applied Polymer Science, Vol. 60, 1193-1199 (1996).

Suitable hydrophilic monomers which may be used separately or in combination, for the silicone hydrogel cores of the present invention non-exclusively include, for example, unsaturated carboxylic acids, such as methacrylic and acrylic acids; acrylic substituted alcohols, such as 2-hydroxyethylmethacrylate, 2-hydroxyethylacrylate (HEMA), and tetraethyleneglycol dimethacrylate (TEGDMA); vinyl lactams, such as N-vinyl pyrrolidone; vinyl oxazolones, such as 2-vinyl-4,4′-dimethyl-2-oxazolin-5-one; and acrylamides, such as methacrylamide and N,N-dimethylacrylamide (DMA). Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Hydrophilic monomers may be incorporated into such copolymers, including, methacrylic acid and 2-hydroxyethyl methacrylamide.

The proportions of the monomers can vary over a large extent. The polymerization mixtures can also include effective amounts of additives, initiators, photoinitiators, and/or catalysts and that the reaction can be conducted in the presence of a diluent. Activation of the initiation of polymerization can be by thermal or photochemical means. The polymerization can occur via any ionic, radical or group transfer mechanism. The punctal plug core can be prepared within a preformed shell or can be formed and subsequently coated with a suitable shell material.

Although in some cases the drug for delivery by the punctal plug can be included in a monomer mixture before polymerization, the drug can be absorbed from solution into the core material before or after formation of the plug comprising a core and shell. For example, in many cases it is difficult to load sufficient quantities of drugs by soaking the silicone hydrogels in aqueous solutions of the drugs. With hydrophobic drugs, the drug's limited solubility in water permits only a small amount of drug to be dissolved in the water, which limits the amount that can be absorbed by the silicone hydrogel from the water solution. In contrast, more hydrophilic drugs, which have a larger solubility in water, generally display a low solubility in a silicone. It has been discovered that in one embodiment of the invention, loading either hydrophobic or hydrophilic drugs into the silicone hydrogel readily occurs by soaking the silicone hydrogel in a non-aqueous solution of the drug, where an organic solvent is used to swell the silicone gel. Non-limiting examples of such organic solvents include ethanol, ethyl acetate, butyl acetate isopropanol, n-propanol, dimethyl sulfoxide (DMSO), methanol, toluene, methylene chloride, and tetrahydrofuran.

In general, the solvent should be one that has a low toxicity, is non-carcinogenic, is non-mutanogenic, or can be removed essentially in total by means commonly employed by those skilled in the art. Many hydrophobic and hydrophilic drugs are soluble in ethanol and so this solvent is conveniently used to load both types of drugs into the gel. The solvents are generally, but not necessarily, removed prior to placement of the punctal plugs into the ocular environment. The solvent can be removed as a volatile off-gassing from the plug, which can be assisted by vacuum, heating, a gas stream, or any combination thereof.

In another embodiment of the invention, drugs can be loaded into the plug by soaking in aqueous solutions. In these cases it is generally necessary to perform the loading over an extended period of time varying from weeks to months. The slow loading is needed when a core material, for example, a silicone hydrogel, do not swell appreciably in water leading to small diffusivity. The small diffusivity, which permits extended release, leads to slow loading where the loading rates are generally comparable to the release rates. Loading in this fashion can be carried out where the punctal plugs are sealed in a container that is used for distribution to a health care professional for insertion into the puncta of a patient. The absorption of the bioactive agent into the core can occur over a long period of time which includes the time of distribution of the punctal plugs. Typically, a use date indicated on such a package including this container would state an initial use date as well as an expiration date such that a sufficient near equilibrium or equilibrium level of the bioactive agent in the core is achieved before use. For example, a punctal plug with a silicone hydrogel core, regardless of the solvent used for loading the bioactive agent into the appliance, can be distributed in an aqueous solution of the bioactive agent.

An alternative embodiment for the loading of the drugs into the silicone hydrogel cores according to the invention is the inclusion of the drugs during the polymerization of monomers and macromers to prepare the silicone hydrogel core. For drugs that display little or no solubility in the monomer mixture, the addition of a solvent, such as those used for swelling of silicone hydrogels, can be included during a solution polymerization where all monomers and the drug are miscible.

A drug in a non-ionic form is desirable in many embodiments of the invention, but a non-ionic form is not required for all embodiments of the invention. Many drugs traditionally supplied in an ionic form can be acquired as, or converted to, the non-ionic equivalent prior to loading in the cores. Any drug that may be absorbed in a core material and is appropriate for introduction to the eye may be used. If desired, a drug can be converted into a more hydrophobic form by protecting a polar functionality such as an acid group, an alcohol, or an amine in a manner where the drug remains protected until released from the core into the aqueous environment of the eye. In general, the drug displays partitioning into the core from an aqueous environment, but the partitioning is not absolute, such that the drug may be released slowly to the aqueous environment, for example, into the tear film adjacent to the core material when a punctal plug is placed in the eye. Multiple drugs may be absorbed into a single core.

When hydrophilic bioactive agents are included in a core, the affinity of the core material for the agent can be enhanced by additional functionality in the core that specifically interacts with the bioactive agent. For the incorporation of ionic drugs, the functionality can be ionic such that the ion pairing of the drug with that functionality occurs. For example, a negatively charged functionality in the silicone hydrogel pairs with a positively charged drug. Other functionalities that can be incorporated into the core are those that can complex a metal ion containing bioactive agent, that can promote two or more specific hydrogen bonding associations with a specific bioactive agent, or that can mimic the biological binding site of the patient for the bioactive agent, for example, the binding site of an enzyme. Other interactions for the enhanced binding to a specific bioactive agent can be used depending upon the nature of the bioactive agent, as can be recognized by one skilled in the art.

In one embodiment, the core can also be loaded with an agent that will affect the rate of diffusion of a hydrophobic drug into the aqueous environment of the eye. For example, when using a relatively hydrophobic drug, the core can be loaded with a hydrophobic molecule of a relatively low molecular weight to augment the relative affinity and permeability of the drug in the core. It is desirable that the hydrophobic molecule is non-toxic and non-inflammatory to the eye if leached from the core into the tear. The hydrophobic molecule can also be a supplement for the treatment of the eye. For example, vitamin E can be included to increase the hydrophobicity of the core and reduce the rate of transfer from the punctal plug to the eye. Alternatively, materials such as surfactants can be incorporated into the plug to enhance the release of extremely hydrophobic compounds into the tear film. In this case, the surfactant will be released into the tear film and this will aid the release of hydrophobic compounds.

In embodiments of the invention, the shell can be made of a polymer that is impermeable to the drug, and the core can be made of a bioabsorbable material. The bioabsorbable material may also be biodegradable. The bioabsorbable material can be, for example, poly lactic co-glycolic acid (PLGA). Other bioabsorbable polyesters, polyorthoesters, polyanhydrides, polyphosphazenes, polyurethanes, or other polymers can be used in embodiments of the invention. In an embodiment using PLGA, the release rate of the drug is controlled by degradation rates of the core, which can have a nearly zero-order release rate.

In an embodiment, the shell and the core may be of materials that are bioabsorbable. The shell can be bioabsorbed at an equal or lesser rate than the core, where the shell remains until the core has effectively released the drug so that the level of drug in a core is insufficient to cause any undesired side effects in the recipient of the punctal plug once the shell can no longer act as an impermeable barrier to the drug. In this embodiment, no removal of the plug is needed at the end of its use when it has been entirely or partially absorbed and in need of replacement by a new punctal if treatment is to continue. An old drug-depleted bioabsorbable punctal plug can be displaced further into the lacrimal canaliculi by insertion of a new plug, allowing the old plug to absorb over a longer period of time than the time needed for essentially complete release of the drug from the plug. Alternatively, once the shell has degraded sufficiently, the blinking process would lead to the plug being swept from the punctum into the tear film.

In another embodiment, the shell can be made of a polymer that is impermeable to the drug, the core can be made of a polymer that is permeable to the drug, and the drug can be dispersed within nano or microparticles embedded in the core polymer. The release of the drug can be moderated by the proportion of particles to the core polymer, and the relative affinity and permeability of the core polymer and particles.

Although not necessary for performance of the punctal plugs of the invention, in some embodiments, the shell is an elastomeric material or can swell in the fluid within the punctum to an extent greater than that of the core material. In these embodiments, little or no cracks or other defects, as in the case of rigid shells, can be promoted by swelling of the core by the fluid.

In another embodiment shown in FIG. 4, the punctal plug 40 can have a three-layer structure with a central core 42, a second shell 46 around the central core 42, and a shell 44 around the second shell 46. The central core 42, which contains the drug to be delivered, can have low permeability with respect to the drug, the second shell 46 can have high permeability with respect to the drug, and the shell 44 can have negligible permeability with respect to the drug. During use, as indicated by arrows in FIG. 4, the drug diffuses in the radial direction from the core 42 and then rapidly diffuses in the axial direction through the second shell 46. This can lead to an effective large drug release to the tears from a drug loaded core polymer, where the low permeability of the core polymer limits the release rates. In an embodiment, the central core can be composed of a bioabsorbable material. In this case, the rate of drug transport is controlled by the absorption or degradation rate of the central core.

In yet another embodiment, as shown in FIG. 5 the punctal plug 50 can have a three-layer structure with a central core 52, a second shell 56 around the central core 52, and a shell layer 54 around a portion of the second shell 56. The central core 52 can contain the drug to be delivered within bioabsorbable or non-bioabsorbable microparticles. The core 52 contains the drug and the second shell 56 around the core 52 controls the drug release rates. The core 52 can have relatively low permeability with respect to the drug, the second shell 56 can have relatively high permeability with respect to the drug, and the shell 54 can have negligible permeability with respect to the drug. In this embodiment, the drug diffuses radially out from the core and the first shell into the tears in the canaliculi, followed by axial diffusion into the tears. For example, the three layer can comprise a HEMA core 52 can have an EGDMA shell 56 around it and a silicon shell 54 covering the EGDMA shell over a portion of the EGDMA shell 56. In this case the drug diffuses radially from the curved surface of the core 52 and then rapidly diffuses axially throught the second shell 56 into the tears. Some drug may diffuse into the canaliculus tissue. This design allows an effective large area of contact between the tears and a drug loaded core 52, where the low permeability of the core 52 polymer limits the release rates. In an embodiment of the invention, the central core can be composed of a bioabsorbable material. In this case, the rate of drug transport is controlled by the absorption or degradation rate of the central core.

In yet another embodiment, the punctal plug 60 can have a structure, as shown in FIG. 6, where a drug containing central core 62 is separated by a void from an impermeable shell 64 concentric with the core. During performance of a method for use of a punctal plug according to this embodiment of the invention, a void between portions of the shell 64 and core 62, allows fluid from the eye can flow into and out of this void, such that a large surface area of the core 62 effectively releases drug to the eye. The core 62 can be either free floating, yet contained, within the shell 64 or attached, as shown, at one or more selected sites to the shell 64. For example, as in FIG. 6, the end opposite the opening to the eye has the shell 64 attached to the suspended core 62 with a void between the core 62 and shell 64 on the axial sides of a cylindrically shaped punctal plug.

Although attachment is shown in FIG. 6 at the end intended to be distal to the eye, attachment can be at any position within the shell that permits the flow of tears about the core. The core can contain bioabsorbable or non-bioabsorbable nano/microparticles, which contain the drug.

The core can be partitioned into multiple features or can have a shape that can increase the surface area that contacts a second layer or is bathed during use by tears. FIG. 7 shows a punctal plug 70 according to an embodiment of the invention where the core 72 is partitioned into four columns that are surrounded by a second shell 76 within an impermeable shell 74, where the core 72 effectively stores the drug and releases it to the second material of a second shell 76 for rapid transport to the eye from the punctal plug 72.

FIG. 8 shows a punctal plug 80 according to an embodiment of the invention, in which the core 82 is partitioned into multiple spheres that reside within a shell 84, where the size of the spheres is greater than the diameter of an orifice through which tear can flow between the partitioned core 82 and the eye. In this embodiment the spheres 84 can be smaller than the opening until they are swollen by water and ultimately tear fluid such that a hollow shell 84 can be readily filled with spheres 82 that ultimately swell to be fixed in the punctal plug 80. Alternately a second shell material with high dispersivity for the drug can be included about the core spheres. The opening can be structured to restrain small spheres or other shaped core particles in a manner such that the surface area or the flow of tears about the particles can be optimized for the controlled delivery of drugs.

A method of treating dry eyes according to an embodiment of the present invention can include inserting any of the punctal plugs described herein into a patient's upper punctum, lower punctum, or both. The punctal plug can help block tear drainage (partially or completely) through the lacrimal canaliculi. Additionally, the drug(s) contained in the punctal plug can be delivered to the patient for an extended period of time. The extended period of time can be, for example, about a month or more. The drug can be any suitable drug known in the art for treatment of an ophthalmic disease or condition, including dry eyes which can be treated using cyclosporine A. Other drugs, such as antibiotics, can be delivered to the eye separately or in conjunction with a drug for dry eyes, such as cyclosporine A. Additives such as nutritional supplements, such as vitamins, minerals, antioxidants, lubricants or any combination thereof, can be delivered by the punctal plugs alone or in addition to one or more drugs. Appropriate additives will depend upon the intended treatment by the punctal plugs and can be appropriately formulated by one skilled in the art.

As up to two punctal plugs can be employed per eye, in one embodiment of the invention directed to a method of use of the novel punctal plugs, a first plug can be placed in the upper punctum to provide one drug formulation and another plug can be placed in the lower punctum to provide a second drug formulation. The drug formulations within the cores of the two plugs can contain one or more common drugs or agents within the formulations or can have no common drugs within the formulations. With common components in the drug formulations of the two plugs, the absolute and relative concentrations of the components can be the same or varied as desired. Each drug formulation in the punctal plug cores can independently contain a single active agent, a plurality of active agents, or no active agents. For example, one punctal plug can contain a combination of drugs and another punctal plug can contain a lubricant. The two punctal plugs can have different core materials and/or different shell materials to achieve a desired effect. For example, one plug can have a core material and design that provides a zero-order release rate while a second plug can be of a core material and design that provides a higher order release rate such that a desired dosage regiment is achieved. One skilled in the art can readily select the drug compositions, punctal plug materials, and punctal plug structures to achieve a desired effect.

### EXAMPLES

### Materials and Methods

Hydroxyl ethylmethacrylate (HEMA), ethylene glycol dimethacrylate (EGDMA), and Azobisisobutylonitrile (AIBN) were purchased from Sigma-Aldrich (St. Louis, Mo.); Cyclosporine A was purchased from LC Labs (Woburn, Mass.); and Silastic® laboratory tubing of three different sizes (ID 0.76 mm, 1.02 mm, and 1.47 mm) was purchased from Dow Corning (Midland, Mich.). The Dulbecco's phosphate buffered saline (PBS) used in the drug release experiments was purchased from Sigma-Aldrich (St Louis, Mo.).

### Composition, Construction and Drug Release of Punctal Plugs

Puncta plugs, as shown in FIG. 3, with only a fraction of the plug length covered with an impermeable shell were prepared. The diameter of the shell was 0.93 mm to ensure a snug fit into the canaliculus and the diameter of the core was 0.51 mm The core of the plug was composed of HEMA and the partial-shell was composed of silicon. The release rate from the plug was varied by varying the cross-linking of the core or by adding a high permeability second shell to have the three layer punctal plugs shown in FIG. 5, using a pure HEMA core, an EGDMA second shell around it and the silicon shell covering a portion of the EGDMA shell. The overall diameter of the three layer plugs was 1.96 mm, the diameter of the EGDMA shell was 1.47 mm and the diameter of the core was 1.02 mm The EGDMA shell had cracks caused by expansion of the HEMA core within the EGDMA shell. The overall diameter (1.9 mm) of the plug design is larger than the diameter of commercial puncta plugs (˜0.9 mm or 1 mm), but the large plugs demonstrate the release properties that can be achieved by punctal plugs of this design.

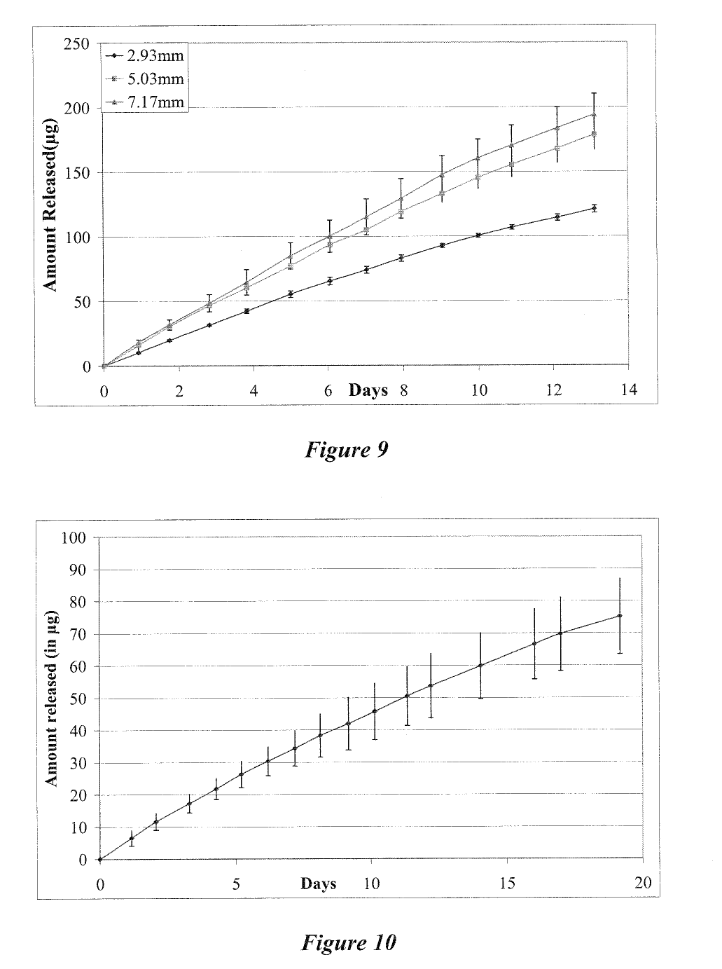

The punctal plugs of the type shown in FIG. 3 had a core with a 0.51 mm diameter of pure HEMA with a fraction of the core coated with a silicon shell with an overall diameter of 0.93 mm. FIG. 9 shows release profiles for three plugs of total length 9.4 mm, 20% drug loading, and overall diameter 0.93 mm but having different ratios of exposed to unexposed portions of the core. The exposed cores are about 2.9 mm, 5.0 mm and 7.2 mm. The drug release profiles show that as the length of exposed core increases, the rate of release increases but the release is not proportional to the length of the exposed core. This behavior is expected because in addition to the radial transport and release from the exposed core, the drug diffuses axially from the core covered with the silicone to the exposed core and through the end of the core, which is identical for the three examined plugs.

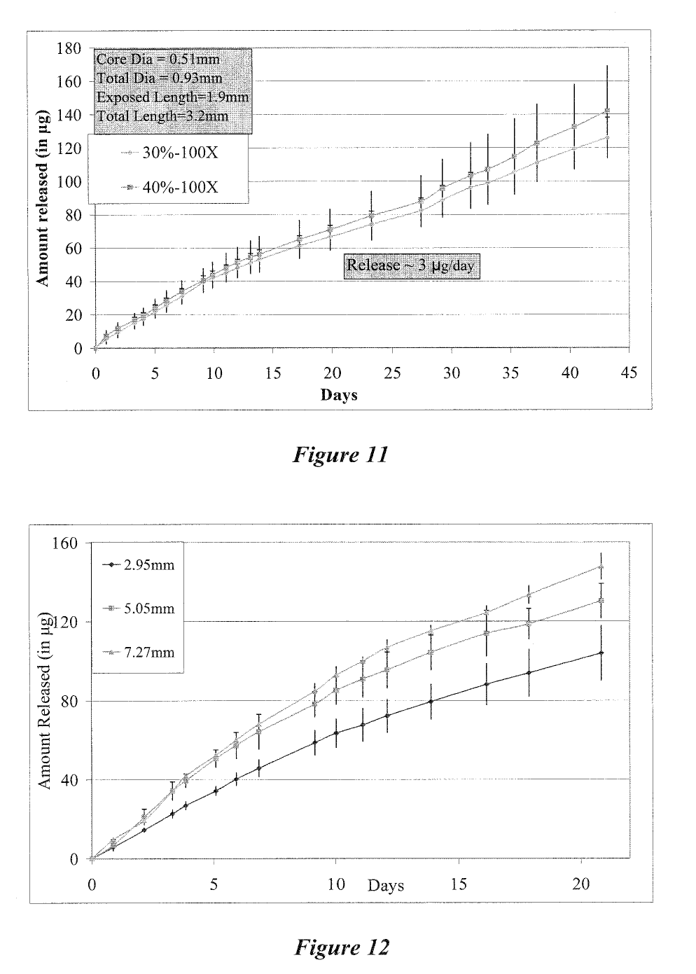

The drug release profiles from a plug 3.4 mm long with 1.87 mm uncovered core are shown in FIG. 10. The drug loading was 20%, and the core and the silicone shell diameters were 0.51 mm and 0.93 mm. The plug releases at about 4 μg/day for about 18 days at close to zero order rates. To increase the release duration the drug loading and the degree of crosslinking were increased. FIG. 11 shows the drug release profiles of three puncta plugs of length 3.4 mm, exposed core length 2 mm, core diameter 0.51 mm and overall diameter 0.93 mm having different drug loadings and cross-linking. Drug loadings are 30% and 40% by wt of cyclosporine in a HEMA core with 22.5% by weight EGDMA cross-linker. The puncta plugs released about 3 μg/day for more than 40 days.

Punctal plugs where the HEMA core was coated with an EGDMA shell and then a fraction is further coated with a silicone shell, in the manner illustrated in FIG. 5, were examined. The overall diameter of the plugs was 1.96 mm, the core diameter was 1.02 mm and the diameter with the EGDMA shell was 1.47 mm. The drug releases through the cracks on the surface of the EGDMA shell that coats the HEMA core. The EGDMA shell can be prepared from a mixture of HEMA and EGDMA and will swell to different degrees depending on the ratio of the two components. When the difference between the swelling of the core and the EGDMA annulus is small fewer cracks result and a slower release is observed. The release profile of plugs with 20% drug loading, an overall length 9.4 mm, and exposed cores of 2.95 mm, 5.05 mm and 7.27 mm are shown in FIG. 12. As above for the cores without an EGDMA shell the release was not proportional to the length of exposed core. The rate of release was about 8.5 μg/day for the exposed length of 5.05 mm. This shows that the EGDMA coatings can be used to control the release rates.

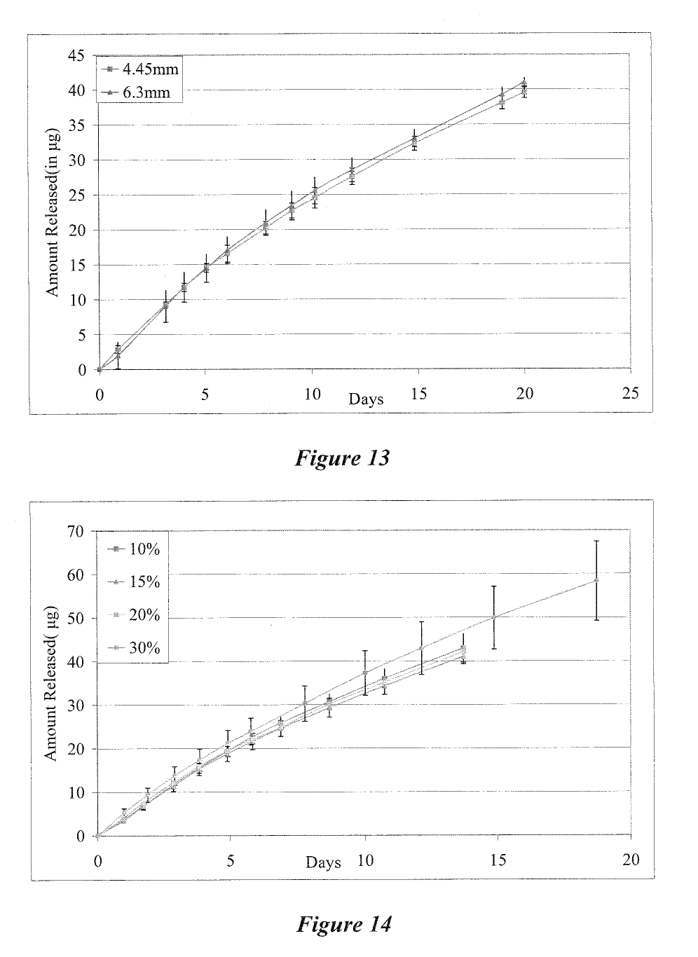

To verify that drug does not diffuse through the silicone shell, drug loaded HEMA cores coated with EGDMA that was entirely covered with a silicone shell were prepared. The diameter of the HEMA core was 1.02 mm, the diameter of EGDMA shell was 1.47 mm and the diameter of silicon shell was 1.96 mm. FIG. 13 shows the release profiles of plugs completely coated with silicon except for the ends of the cylindrical plugs having a core with 20% drug loading and having two different lengths. The release profiles for both lengths was within the error of detection for the first 10 days indicating that no drug was released from through the silicone shell, and that drug released from only the circular ends. FIG. 14 shows the release profile for such plugs with different amounts of drug loading. Each plug released about 3.4 microgram/day for the first 10 days. The profiles are independent of drug loading because the release is controlled by the fluid mass transfer resistance.

The experimental results above did not involve controlled mixing of the fluid in contact with the plugs. The convection generated automatically provided limited mixing. The mixing inside the canaliculi is in fact expected to be very limited and thus these results may be close to the physiological conditions.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A punctal plug, comprising:
a solid core containing at least one drug; and
a drug impermeable shell covering a portion of said core, allowing drug delivery by radial diffusion of said drugs from surfaces of said core in addition to one or both axial ends of said plug.

2. The punctal plug according to claim 1, wherein said plug is cylindrical in shape.

3. The punctal plug according to claim 2, wherein said shell covers said cylindrical plug along a portion of less than said plug's entire length.

4. The punctal plug according to claim 2, wherein said plug has a diameter of about 0.4 to about 1.1 mm and a length of about 1.1 to about 2.0 mm.

5. (canceled)

6. The punctal plug according to claim 1, wherein said core comprises poly hydroxyl ethylmethacrylate.

7. The punctal plug according to claim 1, wherein said core comprises a silicone hydrogel.

8. The punctal plug according to claim 1, wherein said core comprises a bioabsorbable material.

9. The punctal plug according to claim 8, wherein said core comprises poly lactic co-glycolic acid.

10. The punctal plug according to claim 1, wherein said shell comprises a bioabsorbable material.

11. The punctal plug according to claim 1, wherein said core comprises a plurality of nano-particles or micro-particles dispersed in a material wherein said drug is included within said particles.

12. (canceled)

13. The plug according to claim 1, further comprising a second shell of a material with a high diffusivity to said drug disposed between said core and said shell.

14. (canceled)

15. The punctal plug according to claim 13, wherein said second shell comprises poly ethylene glycol dimethacrylate.

16. The punctal plug according to claim 1, wherein said drug is Cyclosporine A or Timolol.

17. The punctal plug according to claim 1, wherein said drug is any ophthalmic drug.

18. The punctal plug according to claim 1, wherein said core further comprises at least one agent selected from the group consisting of nutritional supplements, vitamins, minerals, antioxidants, and lubricants.

19. The punctal plug according to claim 1, wherein said core material is separated from an adjacent portion of said shell by a void.

20. The punctal plug according to claim 1, wherein said core material is partitioned into features or shaped to have a large surface area.

21. The plug according to claim 20, wherein said features are spheres.

22. The plug according to claim 20, wherein said shaped core is an ensemble of a plurality of attached cylinders.

23. A method for treating dry eyes, comprising:
inserting a punctal plug into a lower punctum, an upper punctum, or both; and
allowing said punctal plug to deliver at least one drug to an eye;
wherein said punctal plug comprises a core containing said drug, and a drug impermeable shell covering a portion of said core allowing radial diffusion of said drugs from surfaces of said core in addition to one or both axial ends of said plug.

24-33. (canceled)

34. The method according to claim 23, wherein said punctal plug delivers said drug to the eye for at least a month.

35. The method according to claim 23, wherein said punctal plug delivers said drug to the eye at a rate up to 50 μg/day.

36. A method of making a punctal plug for delivery of a bioactive agent comprising the steps of:
providing a solid core;
providing a shell substantially impermeable to at least one drug where said shell contacts a portion of said core; and
loading said at least one drug from solution to said core.

37. The method of claim 36, wherein said step of providing said core comprises:
filling said shell through an opening of said shell with a liquid comprising at least one monomer; and
polymerizing said monomer.

38. The method of claim 36, wherein said step of providing said shell comprises:
coating said core with a liquid comprising at least one monomer; and
polymerizing said monomer.

39. The method of claim 36, wherein said step of providing said shell comprises:
coating said core with a liquid comprising a polymer in solution; and
removing said solvent from said coating.

40. The method of claim 39, wherein said solution comprises said drug and a non-aqueous solvent.

41. The method of claim 40, further comprising the step of removing said solvent from said loaded core.

42. The method of claim 36, wherein said solution comprises said drug and an aqueous solvent.
